(19) United States
(12) Patent Application Publication
(10) Pub. No.: US 2024/0226040 A1
(43) Pub. Date: Jul. 11, 2024

(54) SOLID PHARMACEUTICAL TABLET

(71) Applicant: Genenxa Inc., Atlanta, GA (US)

(72) Inventors: Max Spielberg, Miami, FL (US); David Johnson, Atlanta, GA (US)

(73) Assignee: Genexa Inc., Atlanta, GA (US)

(21) Appl. No.: 18/619,076

(22) Filed: Mar. 27, 2024

Related U.S. Application Data
(63) Continuation of application No. 18/047,960, filed on Oct. 19, 2022.

(51) Int. Cl. A61K 31/167; A61K 9/20

(52) U.S. Cl. CPC A61K 31/167; A61K 9/2013; A61K 9/2068

(57) ABSTRACT

A tablet includes an active material and a base material comprising carnauba wax and an oil. A tablet formulation can contain an active pharmaceutical ingredient and a base material that includes carnauba wax. The carnauba wax constitutes more than 50% of the base material.

### TECHNICAL FIELD

This application relates to formulations of tablets for the delivery of pharmaceuticals and to solid tablets for oral delivery to patients, people or animals. More particularly, it relates to formulations of solid tablets or caplets having improved characteristics and more natural ingredients.

### BACKGROUND

Oral drug administration has traditionally been the predominant route for drug delivery. A tablet refers to a pharmaceutical oral dosage form (also known as oral solid dosage, or OSD) or a solid unit dosage form. Tablets can be characterized as solid unit dosage forms of medicament or medicaments, accompanied by suitable excipients. These tablets consist of a mixture of active substances and excipients, typically in powder form, which are pressed or compacted into a solid dose. This method is considered the most popular route of drug administration, largely because gastrointestinal physiology allows for greater flexibility in dosage form design compared to most other routes. Consequently, a significant challenge for the pharmaceutical industry in drug development involves producing safe and effective drugs using more natural ingredients.

Like clean food, clean medicines are thought to be minimally processed and free of things like synthetic, or artificial ingredients or preservatives. Although there are many tablet products available, there is a market segment that has been left unaddressed. Organic pharmaceutical tablets have been allusive in the marketplace.

Similar to clean food, clean medicines are perceived as minimally processed and devoid of elements such as synthetic or artificial ingredients and preservatives. Despite the availability of numerous tablet products, there remains an unaddressed market segment. Organic pharmaceutical tablets have been elusive in the marketplace.

Accordingly, there is always a need for an improved solid tablet. It is to this need, among others, that this application is directed.

### SUMMARY

This application provides a tablet comprising: (a) an active material with an ingredient incorporated therein, and (b) a base material primarily consisting of carnauba wax.

In one aspect, the tablet may include an edible oil (e.g., a coating), dextrose, and rice bran-based excipients such as rice bran and rice bran extract.

Another aspect involves a tablet comprising: (a) an active material with a granulated ingredient combined with a carnauba wax base material, and (b) appropriate water-soluble sugars and/or sugar substitutes, and/or rice bran-based excipients like Nu-Rice® and Nu-Flow®. More specifically, one aspect involves a tablet containing an edible oil, dextrose, gum Arabic, and rice bran-based excipients such as rice bran and rice bran extract/hull.

A further aspect encompasses a tablet wherein the hydrophobic material is selected from the group consisting of carnauba wax, beeswax, or a combination thereof.

Another aspect includes a tablet wherein the active ingredient is a pharmaceutical component, a nutritional supplement, or combinations thereof.

Another aspect includes a tablet formulation consisting essentially: (a) an active material comprising an active pharmaceutical ingredient and (b) a base material comprising carnauba wax and an oil, wherein the caramba wax is between 50% and 80% by weight of the base material, and the active agent is greater than 90% by weight of the tablet wherein hardness of the tablet formulation is between 50N and 90N. The active pharmaceutical ingredient can be acetaminophen, ibuprofen. aspirin, naproxen, diphenhydramine, loratadine, pseudoephedrine, dextromethorphan, or guaifenesin.

An additional aspect includes a method of forming a tablet, involving triturating active agents, combining the triturated components with dextrose and rice bran into a mixture, and compressing the mixture into a tablet. The tablet may then be coated with an edible oil.

A subsequent aspect includes a tablet composed of an active ingredient and a base material consisting of carnauba wax, an edible oil coating (e.g., olive oil or sunflower oil), and rice bran or hull. The quantity of carnauba wax exceeds 50%, 60%, 70%, 80%, or 90% of the base material.

Another aspect pertains to a tablet wherein the active agent constitutes more than 90% of the tablet.

An additional aspect involves a tablet wherein the base material primarily consists of carnauba wax and includes an edible oil coating (e.g., palm or sunflower oil), rice bran, rice bran extract, rice hull, and gum (composed of arabic glycoproteins and polysaccharides, predominantly polymers of arabinose and galactose). The hardness of the tablet is between 60N and 100N.

### DETAILED DESCRIPTION

Persons knowledgeable in solid dosage tablet formulations are well-acquainted with various excipients available for formulation. These materials include those listed in the US Food and Drug Administration guide—Generally Recognized as Safe (GRAS).

One embodiment entails a tablet comprising an active ingredient blended with a base material of carnauba wax, along with an edible oil coating. The tablet may incorporate suitable water-soluble sugars, rice bran-based excipients like Nu-Rice® and Nu-Flow®, or rice hulls, and a hydrophobic material. This application reveals carnauba wax as a primary non-active constituent of a tablet that can be effectively compressed with an active ingredient.

The tablet features a formulation of active ingredients integrated into a carnauba wax base material. A narrow particle size distribution may be advantageous for the tablet. The tablet production process may involve direct compression or additional steps (e.g., dry/wet granulation). Granulation is employed to bind powder particles to form larger particles or granules, enhancing tableting quality depending on the formulations. The average particle diameter (volume average particle diameter) within a component group may range between 30-1000 micrometers, 70-500 micrometers, or 120-300 micrometers. For example, the wet granulation process generally includes wet massing of the formula's solid ingredients with a liquid (e.g., water, ethanol, or isopropyl alcohol) to create wet aggregates. Subsequently, the liquid is eliminated from these aggregates to produce dry ones, which are then milled to the desired size. Excessive moisture in the granulation process can yield harder granules.

The solid tablet may possess a hardness from approximately 50 to 110N or about 60 to 90N. Establishing the initial hardness to the aforementioned lower limit or higher facilitates the prevention of tablet damage, such as breakage or chipping, during vibration or handling. Conversely, maintaining hardness at or below the preferred upper limit enhances tablet disintegration.

In one embodiment, tablets might be manufactured through a direct compaction method. This method could involve preparing the excipient base formula, mixing it with one or more active ingredients in a suitable weight ratio to achieve a solid formula, which is then compressed into tablets of the desired size and shape. In certain instances, all required ingredients are combined to ensure uniformity in a suitable blender before the uniform mixture is compressed into tablets. Established tableting processes (e.g., mixing and blending) can be adapted based on specific tablet formulation requirements.

Certain embodiments stipulate initial mixing of active substances with the carnauba wax base material. When active agents are introduced into the carnauba wax base, the material is heated to 180 degrees Celsius to facilitate the integration of the active agents, which are subsequently blended into the tablet matrix.

In one specific embodiment, the tablet formulation consists of an active ingredient, a carnauba wax base material, and organic rice bran (e.g., Nu Rice Bran®), with the carnauba wax constituting more than 50%, 60%, 70%, 80%, or 90% of the base material.

In another embodiment, the tablet's active ingredient is an active pharmaceutical ingredient, a nutritional supplement, or a combination thereof.

In a further embodiment, the active ingredient in the tablet is specifically an active pharmaceutical ingredient.

In another embodiment, a method of forming a tablet as disclosed herein comprises the steps of: preparing a trituration of the active ingredient in a base material; combining this trituration with a mixture containing carnauba wax, organic dextrose, organic rice bran, or rice bran extract; and compressing the combined mixture to form a tablet.

In one embodiment, an edible oil may serve as a coating for the tablet. After the tablet is formed, it can undergo a coating process, if necessary, to enhance the tablet's storage stability. The coating process is not particularly limited, and conventional methods can be employed. For example, one can apply a coating agent (like an aqueous solution of a polymer or an edible oil) to the tablet surface following compression, followed by a drying step.

In another embodiment, the active agent includes an herbal composition effective for managing various disorders. Specifically, the invention pertains to an herbal composition beneficial for managing stress, pain, constipation, among other conditions. Additionally, this herbal composition can be used in the formulation of food supplements, pharmaceuticals, and nutraceuticals intended for addressing disorders associated with metabolic syndrome. A method of treating these disorders is also provided, involving the administration of a therapeutically effective amount of the herbal composition described in this invention.

Examples of suitable analgesics, anti-inflammatories, and antipyretics include, but are not limited to, non-steroidal anti-inflammatory drugs (NSAIDs) such as propionic acid derivatives (e.g., ibuprofen, naproxen, ketoprofen, flurbiprofen, fenbufen, fenoprofen, indoprofen, ketoprofen, fluprofen, pirprofen, carprofen, oxaprozin, pranoprofen, suprofen) and COX inhibitors such as celecoxib; acetaminophen; acetylsalicylic acid; acetic acid derivatives such as indomethacin, diclofenac, sulindac, tolmetin; fenamic acid derivatives such as mefenamic acid, meclofenamic acid, flufenamic acid; biphenylcarboxylic acid derivatives such as diflunisal and flufenisal; and oxicams such as piroxicam, sudoxicam, isoxicam, meloxicam; their isomers; and pharmaceutically acceptable salts and prodrugs thereof.

The percentages of active ingredients present will vary depending on the particular product's desired characteristics. Typically, the base material constitutes at least about 0.001% by weight of the composition, potentially ranging from about 0.001% to about 20%. In some instances, the base material and inactive ingredients collectively contribute from about 0.1% to about 10% by weight. For example, these components might range from about 0.5% to about 10%, from about 1% to about 10%, or from about 1% to about 5% by weight, relative to the composition's total weight. In certain cases, the base material is present in amounts ranging from about 0.001% to about 20% by weight. These quantities might span from about 0.001% to about 0.9%, up to about 1% to about 20% by weight, calculated based on the entire weight of the composition. In other scenarios, the base material exceeds 0.1%, 1%, 2%, 4%, 10%, 20%, 30%, 40%, 50%, 60%, 70%, 80%, 90%, or 95% by weight, contingent on the composition's total weight. In alternate embodiments, the active agent may comprise up to about 90% to 99% of the composition's weight.

The active agent may also exist in the form of pharmaceutically acceptable salts, including but not limited to acidic/anionic or basic/cationic salts. Pharmaceutically acceptable acidic/anionic salts can include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide. Pharmaceutically acceptable basic/cationic salts might include aluminum, benzathine, calcium, chloroprocaine, choline, diethanolamine, ethylenediamine, lithium, magnesium, meglumine, potassium, procaine, sodium, and zinc.

In one embodiment, The tablets may contain common over-the-counter (OTC) medications, which are drugs available without a prescription. These are generally deemed safe for the public and do not require direct supervision by a healthcare professional. The effectiveness of these OTC medications comes from their active pharmaceutical ingredients (APIs). Some of these APIs are found in pain relievers and fever reducers such as Acetaminophen (Tylenol), Ibuprofen (Advil, Motrin), Aspirin, and Naproxen (Aleve). Others are active in medications for coughs, colds, and allergies, including Diphenhydramine (Benadryl), Loratadine (Claritin), Pseudoephedrine, Phenylephrine, Dextromethorphan, and Guaifenesin (Mucinex). Additionally, sleep aids may contain agents like Diphenhydramine (also utilized for its sedative properties) and Doxylamine.

The term “chewing,” in the context of this application, is used to signify that the pulverizing or grinding of a substance occurs through the actions of a patient's or subject's teeth, or gums. This definition does not imply any specific degree of force exerted by the teeth or gums. The essential criterion is that the force applied is sufficient to produce pulverized material, such as granules, particles, or powder, from the tablet through a process of disruption.

The term “pharmaceutical compositions” refers to mixtures comprising carnauba wax, an active ingredient (preferably one, two, or three), and at least one pharmaceutically acceptable excipient.

The term “granules” also encompasses micro-granules. These granules can be administered directly or further processed into various forms, including tablets or mini-tablets.

The term “tablet” encompasses any form of tablet resulting from the compression or compaction of various substances, including powders, granules (obtained through methods such as wet or dry granulation, tableting, or melt extrusion), mini-tablets, micro-granules, and pellets. However, it refers most preferably to a directly compressed tablet.

The term “compressed” pertains to any physical compaction process used in the formation of solid dosage units.

The term “organic,” within the scope of this application, pertains to the methods by which agricultural products are cultivated and processed. Although regulations can differ internationally, in the U.S., organic crops must be grown without utilizing synthetic herbicides, pesticides, fertilizers, or bioengineered genes (GMOs). Tablet formation preferably involves ingredients cultivated without these synthetic substances or GMOs.

### EXAMPLES

### Example 1 (Acetaminophen)

A solid tablet was formulated with acetaminophen as the active ingredient, embedded in a base material primarily composed of carnauba wax. The base material consisted predominantly of carnauba wax and included an edible oil coating (e.g., palm or sunflower oil), rice bran, rice bran extract, rice hull, and gum (composed of arabic glycoproteins and polysaccharides, mainly polymers of arabinose and galactose). The acetaminophen, serving as the active component, was combined with the base material and compressed into tablets, exhibiting satisfactory friability. The active ingredient constituted between 50-90% by weight of the tablet. The base material itself contained over 60% carnauba wax by weight. The remaining composition of the base material included rice bran or hull, rice bran extract, oils, and gum.

### Example 2 (Acetaminophen)

A tablet, pharmaceutical formulation, or pharmaceutical composition was created where the base material comprised more than 65%, 80%, 90%, 95%, or even 98% carnauba wax by weight, calculated on a dry weight basis. This high concentration of carnauba wax in the base material is a defining characteristic of this formulation.

### Example 3 (Ibuprofen)

A tablet, pharmaceutical formulation, or pharmaceutical composition was developed using the same ingredients as described in Example 1, with a significant alteration: the active ingredient, in this case, was ibuprofen instead of acetaminophen. This example demonstrates the versatility of the base material in accommodating different active pharmaceutical ingredients.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A tablet formulation comprising: (a) an active pharmaceutical ingredient, and (b) a base material comprising carnauba wax, wherein the carnauba wax constitutes more than 50% of the base material, and is between 50 and 80% by weight of the base material, with the active agent constituting more than 90% by weight of the tablet, and wherein the hardness of the tablet formulation is between 50N and 90N.

2. A tablet formulation according to claim 1, wherein the carnauba wax is greater than 60% of the base material.

3. A tablet formulation according to claim 1, wherein the carnauba wax is greater than 70% of the base material.

4. A tablet formulation according to claim 1, wherein the carnauba wax is greater than 80% of the base material.

5. A tablet formulation according to claim 1, further comprising an edible oil coating.

6. A tablet formulation according to claim 1, wherein the base material comprises rice bran.

7. A tablet formulation according to claim 1, wherein the active pharmaceutical ingredient is Acetaminophen.

8. A tablet formulation according to claim 1, wherein the pharmaceutical ingredient is Ibuprofen.

9. A tablet formulation according to claim 1, wherein the pharmaceutical ingredient is Aspirin or Naproxen (Aleve).

10. A tablet formulation according to claim 1, wherein the active pharmaceutical ingredient is Diphenhydramine.

11. A tablet formulation according to claim 1, wherein the active pharmaceutical ingredient is Loratadine.

12. A tablet formulation according to claim 1, wherein the active pharmaceutical ingredient is Pseudoephedrine.

13. A tablet formulation according to claim 1, wherein the active pharmaceutical ingredient is Dextromethorphan.

14. A tablet formulation according to claim 1, wherein the active pharmaceutical ingredient is Guaifenesin.

15. A tablet formulation according to claim 1, wherein the tablet is formed by direct compression.

16. A tablet formulation according to claim 1, wherein the active agent is an active pharmaceutical ingredient.

17. A tablet formulation according to claim 1, wherein the active pharmaceutical ingredient is Acetaminophen.

18. A method of forming a tablet, comprising: (a) preparing a base material of carnauba wax and an active pharmaceutical ingredient, wherein the active agent is granulated; (b) combining the base material and the active pharmaceutical ingredient into a mixture; and (c) compressing said combined mixture into a tablet.

19. A method according to claim 18, wherein the mixture includes a sugar, an edible oil, and rice bran.

20. A method according to claim 18, wherein the mixture is a flavoring agent.

21. A method according to claim 18, wherein the carnauba wax is greater than 50% of the base material.

22. A base material for a tablet comprising: (a) carnauba wax and (b) an oil, wherein the carnauba wax is greater than 50% of the base material.

23. A base material for a tablet according to claim 22, wherein the amount of carnauba wax is greater than 70% of the base material.

24. A base material for a tablet according to claim 22, wherein the active ingredient in the tablet is an active pharmaceutical ingredient, a nutritional supplement, or combinations thereof.

25. A base material for a tablet according to claim 22, wherein the amount of carnauba wax is greater than 60% of the base material.

26. A tablet formulation comprising essentially: (a) an active material comprising an active pharmaceutical ingredient, and (b) a base material comprising carnauba wax and an oil, wherein the carnauba wax is between 50 and 80% by weight of the base material, and the active agent is greater than 90% by weight of the tablet, wherein the hardness of the tablet formulation is between 50N and 90N.

27. A tablet formulation according to claim 26, wherein the active pharmaceutical ingredient is acetaminophen.

28. A tablet formulation according to claim 26, wherein the pharmaceutical ingredient is Ibuprofen.

29. A tablet formulation according to claim 26, wherein the pharmaceutical ingredient is Aspirin, or Naproxen.

30. A tablet formulation according to claim 1, wherein the active pharmaceutical ingredient is Diphenhydramine.

31. A tablet formulation according to claim 1, wherein the active pharmaceutical ingredient is Loratadine.

32. A tablet formulation according to claim 1, wherein the active pharmaceutical ingredient is Pseudoephedrine.

33. A tablet formulation according to claim 1, wherein the active pharmaceutical ingredient is Dextromethorphan.

34. A tablet formulation according to claim 1, wherein the active pharmaceutical ingredient is Guaifenesin.
